(19) United States
(12) Patent Application Publication
(10) Pub. No.: US 2025/0125044 A1
(43) Pub. Date: Apr. 17, 2025

(54) CAREGIVING ROBOT AND CAREGIVING SYSTEM AND METHOD EMPLOYING THE SAME

(71) Applicant: Delta Electronics Int’l (Singapore) Pte Ltd, Singapore (SG)

(72) Inventors: Huanyue Liao, Singapore (SG); Tzu-Yi Hung, Singapore (SG); Li Han Chen, Singapore (SG); Qing Liu, Singapore (SG)

(21) Appl. No.: 18/910,293

(22) Filed: Oct. 9, 2024

Related U.S. Application Data
(60) Provisional application No. 63/543,772, filed on Oct. 12, 2023.

(51) Int. Cl. G16H 40/67; B25J 5/00; B25J 11/00; G10L 13/02; G10L 15/00; G10L 15/06; G10L 15/183; G10L 15/22; G16H 50/30; G16H 80/00

(52) U.S. Cl. CPC G16H 40/67; B25J 5/00; B25J 11/009; G10L 13/02; G10L 15/005; G10L 15/063; G10L 15/183; G10L 15/22; G16H 50/30; G16H 80/00; G10L 2015/223

(57) ABSTRACT

A caregiving robot and a caregiving system and method employing the same are provided. The caregiving robot includes a movement module, an interaction module and a control module. The interaction module receives an input instruction from the caregiver, and the input instruction includes an identity information and a target location information of the care recipient and a caregiving task information. During execution of the caregiving task, the control module controls the movement module to make the caregiving robot move to a target location according to the target location information. When the caregiving robot arrives at the target location, the control module controls the interaction module to interact with the care recipient according to the caregiving task information so as to collect health status input from the care recipient, and generates a status report accordingly. The status report includes health status information and status evaluation information of the care recipient.

### CROSS-REFERENCE TO RELATED APPLICATION

This application claims the benefit of U.S. Provisional Application No. 63/543,772 filed on Oct. 12, 2023 and entitled “HEALTH REMOTE CHECK-IN ROBOTIC SYSTEM INTEGRATED WITH A LARGE LANGUAGE MODEL FOR ENHANCING ELDERLY CARE”. The entire contents of the above-mentioned patent application are incorporated herein by reference for all purposes.

### FIELD OF THE INVENTION

The present disclosure relates to a caregiving robot and a caregiving system and method employing the same, and more particularly to a caregiving robot and a caregiving system and method employing the same for reducing the burden on caregivers.

### BACKGROUND OF THE INVENTION

With the continuous increasing proportion of the global elderly population, demographic changes would bring a lot of challenges to the healthcare and caregiving fields. Routine health status checks are crucial to ensuring the safety and well-being of elderly individuals, especially for those living alone.

Conventionally, routine health status checks are executed manually (e.g., by staff from caregiving institutions or social welfare service centers) through on-site visits or telephone interviews to regularly check the health status of elderly individuals. However, this approach has certain limitations. Relying on human labor may lead to omissions or errors due to human factors, and the labor-intensive nature of this work further increases the burden on the overall caregiving system.

### SUMMARY OF THE INVENTION

The present disclosure provides a caregiving robot and a caregiving system and method employing the same to in order to overcome the drawbacks of conventional technologies. Through the caregiving robot and the caregiving system and method employing the same of the present disclosure, the workload of caregivers can be reduced, and the demand for human resources in caregiving can be lowered, thereby alleviating the burden on the overall caregiving system.

In accordance with an aspect of the present disclosure, a caregiving robot is provided. The caregiving robot includes a movement module, an interaction module and a control module. The movement module is configured to enable the caregiving robot to move. The interaction module is configured to interact with a care recipient and a caregiver and receive one or more input instructions from the caregiver, and the input instruction at least includes an identity information of the care recipient, a target location information of the care recipient and a caregiving task information. The control module is electrically connected to the movement module and the interaction module and configured to control the movement module and the interaction module. The control module is configured to perform a caregiving task according to the input instruction(s) received by the interaction module. During execution of the caregiving task, the control module is configured to control the movement module to make the caregiving robot move to a target location where the care recipient is located according to the target location information. When the caregiving robot arrives at the target location, the control module is configured to control the interaction module to interact with the care recipient according to the caregiving task information so as to collect health status input from the care recipient. The control module is further configured to generate a status report according to the health status input, and the status report includes health status information and status evaluation information of the care recipient.

### BRIEF DESCRIPTION OF THE DRAWINGS

FIG. 1 is a schematic block diagram illustrating a caregiving robot according to an embodiment of the present disclosure;

FIG. 2 exemplifies the detailed structure of the caregiving robot 1 of FIG. 1;

FIG. 3 is a schematic flow chart illustrating a first mode of the caregiving task according to an embodiment of the present disclosure;

FIG. 4 is a schematic flow chart illustrating a variant of the first mode of the caregiving task shown in FIG. 3;

FIG. 5 is a schematic flow chart illustrating sub-steps of the step of the caregiving robot engaging in the conversation with the care recipient shown in FIG. 3 and FIG. 4;

FIG. 6 is a schematic flow chart illustrating a second mode of the caregiving task according to an embodiment of the present disclosure;

FIG. 7 is a schematic flow chart illustrating a variant of the second mode of the caregiving task shown in FIG. 6; and

FIG. 8 is a schematic block diagram illustrating a caregiving system according to an embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only.

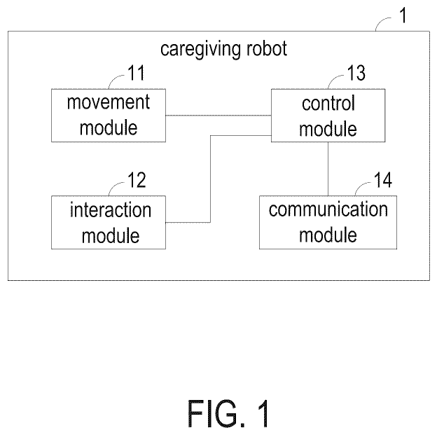

Please refer to FIG. 1. FIG. 1 is a schematic block diagram illustrating a caregiving robot according to an embodiment of the present disclosure. As shown in FIG. 1, the caregiving robot 1 includes a movement module 11, an interaction module 12 and a control module 13. The movement module 11 is configured to enable movement of the caregiving robot 1, and the movement module 11 may include wheels, tracks, or other movement components driven by a motor. The interaction module 12 is configured to receive one or more input instructions from a caregiver, and the input instruction at least includes an identity information of a care recipient, a target location information of the care recipient and a caregiving task information to be performed by the caregiving robot 1. The control module 13 is electrically connected to the movement module 11 and the interaction module 12, and is configured to control the movement module 11 and the interaction module 12. The control module 13 may be implemented with a suitable processor or microcontroller. In the caregiving method proposed in the present disclosure, the interaction module 12 receives the input instruction from the caregiver, and the control module 13 controls the caregiving robot 1 to perform a caregiving task according to the input instruction received by the interaction module 12. During execution of the caregiving task, according to the target location information of the care recipient, the control module 13 is configured to control the movement module 11 to make the caregiving robot 1 move to a target location where the care recipient is located. As the caregiving robot 1 arrives at the target location, the control module 13 is configured to control the interaction module 12 to interact with the care recipient according to the caregiving task information so as to collect health status input from the care recipient. The control module 13 is further configured to generate a status report according to the collected health status input, and the status report includes health status information and status evaluation information of the care recipient. In an embodiment, the control module 13 may also provide relevant recommendations in the status report for the caregiver's reference.

From the above, the caregiving robot 1 can independently go to the location of the care recipient and check the health status of the care recipient. Therefore, routine health checks can be performed by the caregiving robot 1. Accordingly, the workload of caregivers can be reduced, and the demand for human resources in caregiving can be lowered, thereby alleviating the burden on the overall caregiving system.

In an embodiment, the caregiving robot 1 further includes a communication module 14 electrically connected to and controlled by the control module 13. The communication module 14 is configured to communicate with a management device of the caregiver and to transmit the status report to the management device (e.g., the mobile phone of caregivers and/or a designated server) via the communication. The communication module 14 supports wireless transmission, such as via Wi-Fi and/or cellular networks.

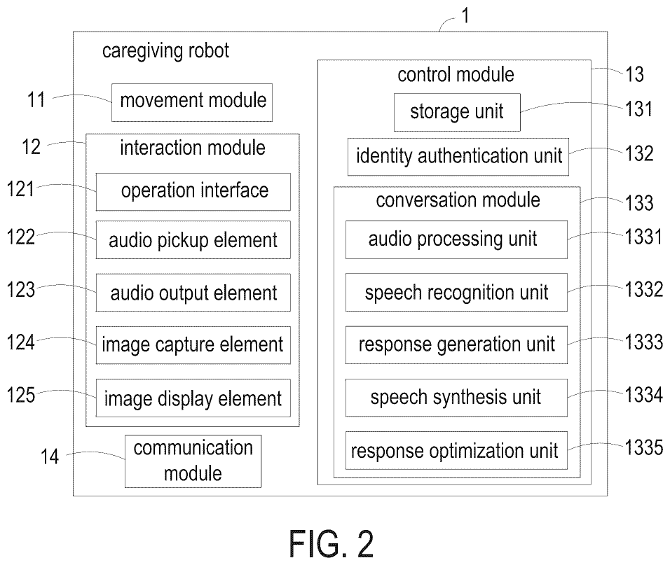

Please refer to FIG. 2. FIG. 2 exemplifies the detailed structure of the caregiving robot 1 of FIG. 1. In an embodiment, the interaction module 12 of the caregiving robot 1 further includes an operation interface 121. The operation interface 121 allows the caregiver to provide the input instruction for controlling the caregiving robot 1. The operation interface 121 may include hardware and/or software interfaces, such as an application, buttons, a keyboard, a touchscreen and/or a touchpad, but not limited thereto.

In addition, the interaction module 12 may incorporate suitable interaction elements according to the required interaction capabilities. For example, the interaction module 12 may include an audio pickup element 122 (for example but not limited to a microphone), an audio output element 123 (for example but not limited to a speaker), an image capture element 124 (for example but not limited to a camera), and/or an image display element 125 (for example but not limited to a screen). The audio pickup element 122 is configured to capture sounds and generate a sound signal accordingly. The audio output element 123 is configured to play sound according to a received sound signal. The image capture element 124 is configured to capture images and generate an image signal accordingly. The image display element 125 is configured to display images according to a received image signal. It is noted that although the operation interface 121, audio pickup element 122, audio output element 123, image capture element 124, and image display element 125 are described as separate components for clarity, they can share the same hardware when their functions overlap. Taking a touchscreen as an example, the touchscreen may serve as both the operation interface 121 for receiving the input instruction from the caregiver and the image display element 125 for displaying images.

Moreover, the caregiving robot 1 supports interaction in multiple languages and can interact with the care recipient in the same language that the care recipient uses. For example, the control module 13 may determine the language used by a person according to information gathered during the interaction between the interaction module 12 and the person (e.g., through collecting the sound made by the person) and control the interaction module 12 to communicate with the person in that language.

In an embodiment, the control module 13 of the caregiving robot 1 includes a storage unit 131. The storage unit 131 is configured to store data required for the caregiving robot 1 to perform the caregiving task. Further, the storage unit 131 may also store the status report generated by the control module 13. The data required for the caregiving robot 1 to perform the caregiving task may include a floor plan of the environment where the caregiving robot 1 is located, and the residence address of the care recipient, but not limited thereto. Additionally, the storage unit 131 may further be configured to store the software executed by the control module 13. The storage unit 131 may include a hard drive or memory, but not exclusively. In addition, in an embodiment, the storage unit 131 may be connected to a cloud server and backup data on the cloud server.

According to the input instruction from the caregiver, the caregiving robot 1 may perform the caregiving task once, periodically (at regular intervals or scheduled times), or when a certain condition is met. For example, the caregiving robot 1 may perform the caregiving task on a regular basis (e.g., daily) or automatically performs the caregiving task when the care recipient does not respond to a contact attempt from the caregiver (e.g., via on-site visits or phone calls), in order to promptly check the health status of the care recipient. Moreover, the caregiving task may be combined with other types of tasks, such as meal delivery, medicine delivery, or package delivery. In addition, the operation interface 121 may be operated by the care recipient to input instructions, which allows the care recipient to actively control the caregiving robot 1 to perform the caregiving task when needed, thereby reflecting the health status of the care recipient to the caregiver through the caregiving robot 1.

In an embodiment, the control module 13 further includes an identity authentication unit 132 configured to authenticate the identities of the caregiver and the care recipient. For instance, after the identity authentication unit 132 authenticates the identity of the caregiver, the control module 13 grants the caregiver permission to view the related information of care recipient and input instructions through the operation interface 121. On the other hand, during the execution of the caregiving task, when the caregiving robot 1 arrives at the target location where the care recipient is located, the identity authentication unit 132 authenticates the identity of the care recipient through the interaction module 12 to ensure that the identity of the care recipient matches the identity information included in the input instruction. The identity authentication unit 132 may utilize various kinds of identity authentication manners, for example but not limited to facial recognition, fingerprint recognition, barcodes, QR codes and/or RFID (radio-frequency identification), and the specific implementation of the identity authentication unit 132 may be adjusted according to actual requirements. Further, the specific actions of the identity authentication unit 132 depend on the identity authentication manner used. For example, when the facial recognition is used for identity authentication, the identity authentication unit 132 controls the image capture element 124 of the interaction module 12 to capture the facial image of a person and compares the captured facial image with the image stored in the database to verify the identity of the person.

The caregiving task performed by the caregiving robot 1 may include different modes, and parameters (e.g., task content and execution time) set for different modes may be different, which allows the caregiving robot 1 to perform different actions in different modes. Typically, when the caregiver operates the operation interface 121 of the interaction module 12 to input instructions, the caregiver may also specify the mode of the caregiving task. In addition, by using the communication module 14 to enable the caregiving robot 1 to communicate with the management device of the caregiver, the caregiver can remotely control the caregiving robot 1 to perform the caregiving task in a specific mode. Moreover, the caregiving robot 1 may be set to automatically perform a specific mode when a certain condition is met. A first mode and a second mode of the caregiving task would be exemplified as follows.

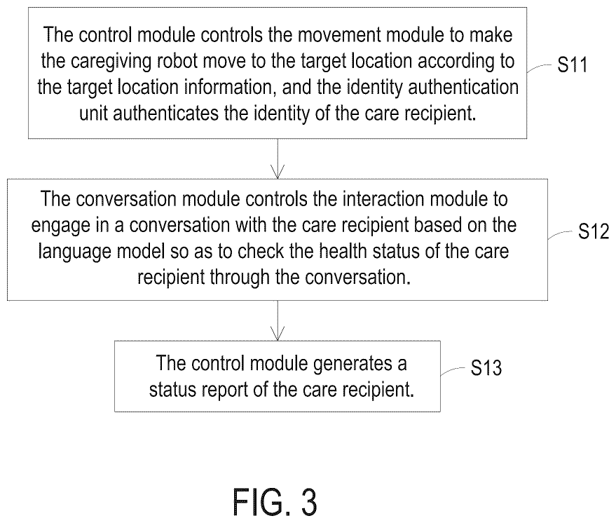

Please refer to FIG. 3 with FIG. 2. FIG. 3 is a schematic flow chart illustrating a first mode of the caregiving task according to an embodiment of the present disclosure. In this embodiment, the control module 13 of the caregiving robot 1 further includes a conversation module 133, which utilizes a language model to generate response content according to the human's speech, thereby realizing the conversation between the caregiving robot 1 and humans. The language model may include a large language model (LLM) based on neural networks, but not limited thereto. The first mode of the caregiving task includes the following steps. First, in step S11, the control module 13 controls the movement module 11 to make the caregiving robot 1 move to the target location where the care recipient is located according to the target location information of the care recipient, and the identity authentication unit 132 authenticates the identity of the care recipient. Then, in step S12, the conversation module 133 controls the interaction module 12 to engage in a conversation with the care recipient based on the language model so as to check the health status of the care recipient through the conversation and collect the health status input from the care recipient. Finally, in step S13, the control module 13 generates a status report of the care recipient. For example, the control module 13 may generate the status report according to the content of the conversation between the caregiving robot 1 and the care recipient.

Consequently, the caregiving robot 1 can independently engage in conversation with the care recipient and check the health status of the care recipient. Accordingly, the burden on caregivers is further reduced.

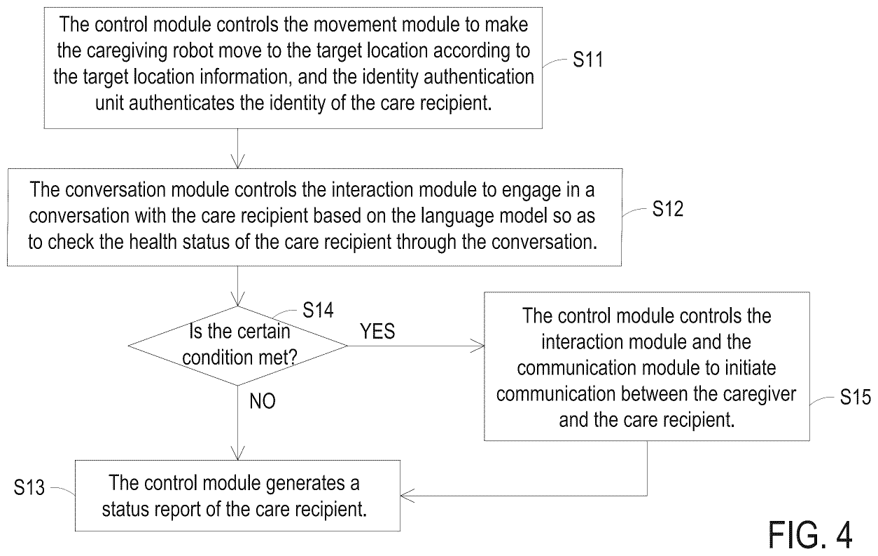

Please refer to FIG. 4 with FIG. 2. FIG. 4 is a schematic flow chart illustrating a variant of the first mode of the caregiving task shown in FIG. 3. In FIG. 4, the steps corresponding to those of FIG. 3 are designated by the same numeral references, and thus the detailed descriptions thereof are omitted herein. In an embodiment, the first mode further includes steps S14 and S15. After performing step S12 in which the caregiving robot 1 engages in the conversation with the care recipient, in step S14, the control module 13 determines whether a certain condition is met according to the content of the conversation. If the determination result of step S14 is positive, the control module 13 determines that there is a need for the care recipient to have a conversation with the caregiver. Accordingly, step S15 is performed to control the interaction module 12 and the communication module 14 to initiate communication between the caregiver and the care recipient, and thus the caregiver is allowed to check the health status of the care recipient via the communication. Conversely, if the determination result of step S14 is negative, then step S13 is performed to generate the status report. The said certain condition may include that the care recipient actively requests to communicate with the caregiver or the health status of the care recipient needs to be further checked by the caregiver (which may be determined by the caregiving robot 1 according to the content of the conversation between the caregiving robot 1 and the care recipient), for example but not limited to the situation of critical symptoms.

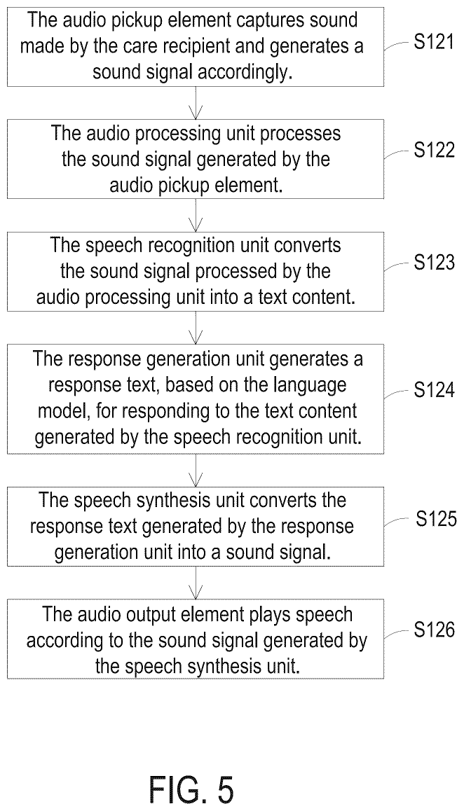

Please refer to FIG. 5 with FIG. 2. FIG. 5 is a schematic flow chart illustrating sub-steps of the step of the caregiving robot engaging in the conversation with the care recipient (i.e., step S12) shown in FIG. 3 and FIG. 4. In some embodiments, the conversation module 133 of the control module 13 includes an audio processing unit 1331, a speech recognition unit 1332, a response generation unit 1333, and a speech synthesis unit 1334. The audio processing unit 1331 is connected to the audio pickup element 122 of the interaction module 12 and is configured to process the sound signal (including but not limited to noise cancellation) generated by the audio pickup element 122. The speech recognition unit 1332 is connected to the audio processing unit 1331 and is configured to convert the sound signal processed by the audio processing unit 1331 into a text content. For example, the speech recognition unit 1332 may utilize the Whisper model to convert the sound signal into the text content. The response generation unit 1333 is connected to the speech recognition unit 1332 and is configured to generate a response text, based on the language model, for responding to the text content generated by the speech recognition unit 1332. The speech synthesis unit 1334 is connected to the response generation unit 1333 and the audio output element 123 of the interaction module 12. The speech synthesis unit 1334 is configured to convert the response text generated by the response generation unit 1333 into a sound signal. The audio output element 123 is configured to play speech according to the sound signal generated by the speech synthesis unit 1334.

In this embodiment, regarding the first mode of the caregiving task, the step of the caregiving robot engaging in the conversation with the care recipient includes the following sub-steps. Firstly, in step S121, the audio pickup element 122 captures sound made by the care recipient and generates a sound signal accordingly. Then, in step S122, the audio processing unit 1331 processes the sound signal generated by the audio pickup element 122. Next, in step S123, the speech recognition unit 1332 converts the sound signal processed by the audio processing unit 1331 into a text content. Afterwards, in step S124, the response generation unit 1333 generates a response text, based on the language model, for responding to the text content generated by the speech recognition unit 1332. Then, in step S125, the speech synthesis unit 1334 converts the response text generated by the response generation unit 1333 into a sound signal. Finally, in step S126, the audio output element 123 plays speech according to the sound signal generated by the speech synthesis unit 1334.

In an embodiment, the response generation unit 1333 includes an AI (artificial intelligence) chatbot, for example but not limited to ChatGPT. Through this AI chatbot, the caregiving robot 1 can conduct a natural conversation with the care recipient, following the care recipient's responses and freely steering the conversation, without being limited to preset questions and answers. This allows the care recipient to have a better conversational experience. For example, during the conversation, the caregiving robot 1 can inquire about the health status of the care recipient in a caring and natural way, guiding the care recipient to describe the health condition without being overly abrupt or formal. Additionally, the caregiving robot 1 may ask follow-up questions based on the care recipient's descriptions of their health to gain more detailed insights, which leads to a more comprehensive assessment of the health status of the care recipient, and the caregiving robot 1 may even offer the care recipient health suggestions. Moreover, in addition to checking the health status, the caregiving robot 1 may engage in a casual conversation with the care recipient about their daily life, thus providing companionship and contributing to the mental well-being of the care recipient.

In addition, the caregiving robot 1 supports the conversation in multiple languages and can interact with the care recipient in the same language that the care recipient uses. In an embodiment, when the identity authentication unit 132 of the control module 13 completes the authentication of the identity of the care recipient, the conversation module 133 knows the language that the care recipient uses according to the personal information of the care recipient and automatically uses that language to communicate with the care recipient. In an embodiment, when the care recipient speaks to the caregiving robot 1, the conversation module 133 may automatically identify the language being spoken and use that language to interact with the care recipient.

Furthermore, to make the response of the caregiving robot 1 to the care recipient more appropriate, in an embodiment, the conversation module 133 further includes a response optimization unit 1335. The response optimization unit 1335 may adjust the response of the caregiving robot 1 to the care recipient by training the language model. For example, based on the personal information of the care recipient, such as gender, personality, age and preferences, the response optimization unit 1335 may train the language model to configure the role and wordings used by the caregiving robot 1 during the conversation. Further, the response optimization unit 1335 may also control the speech synthesis unit 1334 to configure the tone and pitch of the caregiving robot 1 during the conversation. Additionally, the response optimization unit 1335 may limit the types of information or the way of describing that the response generation unit 1333 can provide when generating response text, thereby preventing the caregiving robot 1 from delivering confidential data or incorrect information or causing negative emotional reactions in the care recipient.

In an embodiment, the speech recognition unit 1332, the response generation unit 1333, the speech synthesis unit 1334 and/or the response optimization unit 1335 of the conversation module 133 may be capable of supporting multiple languages. The control module 13 may select the corresponding unit to recognize and generate interactive information according to the selected or identified language.

Please refer to FIG. 2 again. In the second mode of the caregiving task, after the caregiving robot 1 arrives at the target location where the care recipient is located and completes the identity authentication, the caregiving robot 1 initiates communication between the caregiver and the care recipient, and thus the caregiver is allowed to check the health status of the care recipient via the communication. After the communication ends, the caregiving robot 1 generates a status report according to the above process. The form of the communication between the caregiver and the care recipient depends on the implementation of the interaction module 12. For example, if the interaction module 12 includes the audio pickup element 122 and the audio output element 123, the interaction module 12 allows the caregiver to communicate with the care recipient via voice call. If the interaction module 12 includes the audio pickup element 122, the audio output element 123, the image capture element 124 and the image display element 125, the interaction module 12 allows the caregiver to communicate with the care recipient via video call.

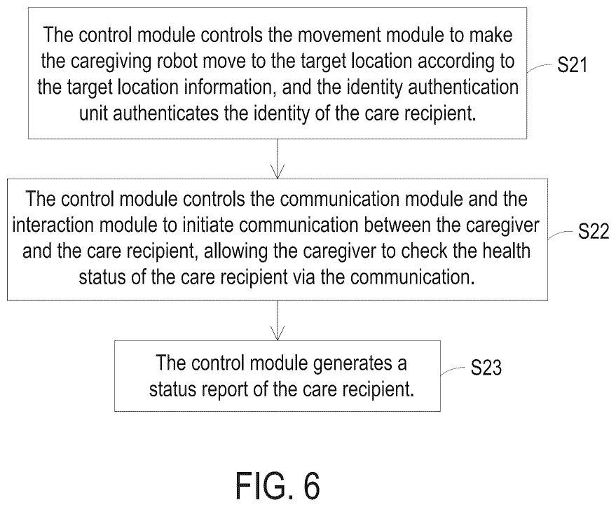

Please refer to FIG. 6 with FIG. 2. FIG. 6 is a schematic flow chart illustrating a second mode of the caregiving task according to an embodiment of the present disclosure. The second mode of the caregiving task includes the following steps. Firstly, in step S21, the control module 13 controls the movement module 11 to make the caregiving robot 1 move to the target location where the care recipient is located according to the target location information, and the identity authentication unit 132 authenticates the identity of the care recipient. Then, in step S22, the control module 13 controls the communication module 14 and the interaction module 12 to initiate communication between the caregiver and the care recipient. Therefore, the caregiver is allowed to check the health status of the care recipient via the communication, and thus the health status input of the care recipient is collected. Finally, in step S23, the control module 13 generates a status report of the care recipient. For instance, the control module 13 may generate the status report according to the communication between the caregiver and the care recipient.

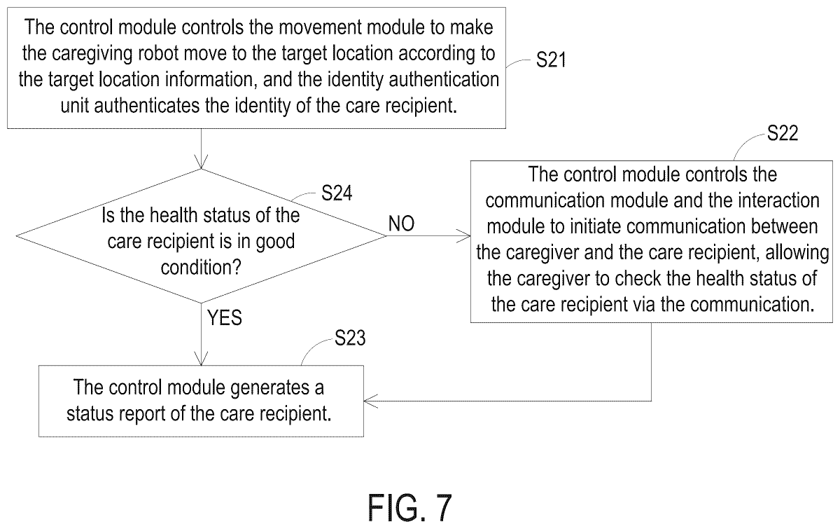

Please refer to FIG. 7 with FIG. 2. FIG. 7 is a schematic flow chart illustrating a variant of the second mode of the caregiving task shown in FIG. 6. In FIG. 7, the steps corresponding to those of FIG. 6 are designated by the same numeral references, and thus the detailed descriptions thereof are omitted herein. In the embodiment shown in FIG. 7, step S24 is performed after step S21. In step S24, the control module 13 controls the interaction module 12 to ask the care recipient whether their health status is in good condition (i.e., whether it meets a preset condition). If the care recipient answers yes (i.e., their health status is in good condition), step S23 is performed to let the control module 13 generate the status report which records that the health status of the care recipient is in good condition. Conversely, if the care recipient answers no (i.e., their health status is not in good condition), step S22 is performed to allow the caregiver to further check the health status of the care recipient through the communication and provide appropriate assistance. Under this circumstance, the content of the status report generated by the control module 13 may vary depending on the actual settings. For example, the status report may record that the health status of the care recipient is not in good condition and/or record the content of the communication between the caregiver and the care recipient.

In practical applications, the specific wording used in step S24 for asking the care recipient about their health status may be adjusted according to actual requirements. For example, the question could inquire about mental well-being, dietary conditions and/or the status of specific body parts. The caregiver may modify the content of the question according to actual requirements.

In addition, in step S24, the interaction module 12 of the caregiving robot 1 may ask the care recipient through voice and/or text, and the care recipient may respond verbally or respond through operating the operation interface 121. For instance, if the operation interface 121 of the interaction module 12 is a touchscreen, the operation interface 121 may display the inquiry text along with “Yes” and “No” buttons, and the care recipient may answer by touching the corresponding button on the operation interface 121 to indicate whether their health status is in good condition.

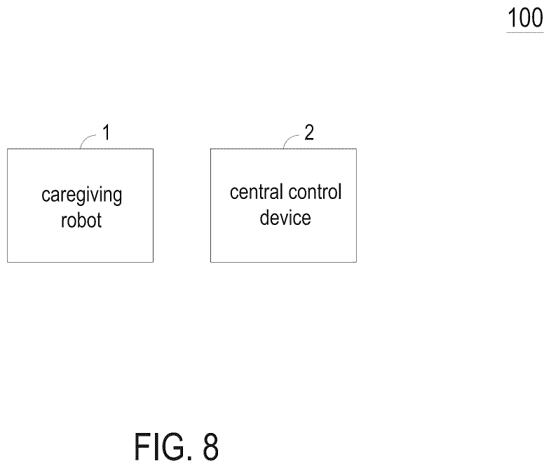

Please refer to FIG. 8. FIG. 8 is a schematic block diagram illustrating a caregiving system according to an embodiment of the present disclosure. As shown in FIG. 8, the caregiving system 100 includes the caregiving robot 1 and a central control device 2 in communication with each other, and the central control device 2 is above to control the actions of the caregiving robot 1. The caregiver may operate the central control device 2 to assign tasks to the caregiving robot 1. For example, the caregiver may operate the central control device 2 to send a control signal to the caregiving robot 1, thereby enabling the caregiving robot 1 to execute the caregiving task according to the control signal. In addition, in an embodiment, the caregiving system 100 may include a plurality of caregiving robots 1 controlled by the central control device 2, and the caregiver can manage and dispatch the plurality of caregiving robots 1 through the central control device 2.

According to the above embodiments, the caregiving robot and the caregiving system and method employing the same may be applied in locations with a high density of individuals requiring care (e.g., medical institutions, nursing homes and caregiving centers), thereby alleviating the burden on caregivers.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A caregiving robot, comprising:
a movement module, configured to enable the caregiving robot to move;
an interaction module, configured to interact with a care recipient and a caregiver and receive an input instruction from the caregiver, wherein the input instruction at least comprises an identity information of the care recipient, a target location information of the care recipient and a caregiving task information; and
a control module, electrically connected to the movement module and the interaction module and configured to control the movement module and the interaction module, wherein the control module is configured to perform a caregiving task according to the input instruction received by the interaction module, during execution of the caregiving task, the control module is configured to control the movement module to make the caregiving robot move to a target location where the care recipient is located according to the target location information, and when the caregiving robot arrives at the target location, the control module is configured to control the interaction module to interact with the care recipient according to the caregiving task information so as to collect health status input from the care recipient, wherein the control module is further configured to generate a status report according to the health status input, and the status report comprises health status information and status evaluation information of the care recipient.

2. The caregiving robot according to claim 1, wherein according to the input instruction from the caregiver, the control module performs the caregiving task once, periodically or when a certain condition is met, and the certain condition comprises that the care recipient does not respond to a contact attempt from the caregiver.

3. The caregiving robot according to claim 1, wherein the interaction module comprises an operation interface configured for the caregiver to operate to provide the input instruction.

4. The caregiving robot according to claim 3, wherein the operation interface is further configured for the care recipient to operate to input an instruction, and the control module is further configured to perform the caregiving task according to the instruction input by the care recipient through the operation interface.

5. The caregiving robot according to claim 1, wherein the control module is configured to automatically identify a language used by the care recipient according to an interaction between the interaction module and the care recipient, and the control module controls the interaction module to continue the interaction with the care recipient in the language.

6. The caregiving robot according to claim 1, wherein the control module knows a language used by the care recipient according to the identity information of the care recipient, and the control module is configured to control the interaction module to interact with the care recipient in the language during the execution of the caregiving task.

7. The caregiving robot according to claim 1, wherein the control module comprises an identity authentication unit configured to authenticate identities of the caregiver and the care recipient, in the caregiving task, when the caregiving robot arrives at the target location, the identity authentication unit authenticates the identity of the care recipient to ensure that the identity of the care recipient matches the identity information included in the input instruction.

8. The caregiving robot according to claim 1, further comprising a communication module electrically connected to the control module, wherein the communication module is configured to communicate with a management device of the caregiver, the interaction module comprises an audio pickup element and an audio output element, the audio pickup element is configured to capture sound and generate a sound signal accordingly, and the audio output element is configured to play sound according to a received sound signal.

9. The caregiving robot according to claim 8, wherein in the caregiving task, when the caregiving robot arrives at the target location, the control module is configured to control the communication module and the interaction module to initiate communication between the caregiver and the care recipient, which allows the caregiver to check a health status of the care recipient via the communication.

10. The caregiving robot according to claim 9, wherein the communication between the caregiver and the care recipient is a video call, the interaction module further comprises an image capture element and an image display element, the image capture element is configured to capture an image of the care recipient and generate an image signal accordingly, and the image display element is configured to display an image according to an image signal received by the communication module.

11. The caregiving robot according to claim 9, wherein in the caregiving task, when the caregiving robot arrives at the target location, the control module is configured to control the communication module and the interaction module to initiate the communication between the caregiver and the care recipient if the control module knows that a health status of the care recipient doesn't meet a preset condition through the interaction module.

12. The caregiving robot according to claim 8, wherein the interaction module further comprises a conversation module configured to generate response content for responding to human's speech based on a language model; in the caregiving task, when the caregiving robot arrives at the target location, the conversation module is configured to control the interaction module to engage in a conversation with the care recipient based on the language model so as to check a health status of the care recipient through the conversation.

13. The caregiving robot according to claim 12, wherein during the conversation between the caregiving robot and the care recipient, the control module is configured to control the interaction module and the communication module to initiate communication between the caregiver and the care recipient when a certain condition is met, and the certain condition comprises that the care recipient actively requests to communicate with the caregiver and/or the control module determines that the health status of the care recipient is at risk.

14. The caregiving robot according to claim 12, wherein the conversation module comprises an artificial intelligence chatbot, and the artificial intelligence chatbot is configured to generate the response content according to speech of the care recipient and guide the care recipient to describe the health status.

15. The caregiving robot according to claim 12, wherein the conversation module comprises:
an audio processing unit, connected to the audio pickup element of the interaction module, and configured to process the sound signal generated by the audio pickup element;
a speech recognition unit, connected to the audio processing unit and configured to convert the sound signal processed by the audio processing unit into a text content;
a response generation unit, connected to the speech recognition unit and configured to generate a response text, based on the language model, for responding to the text content generated by the speech recognition unit; and
a speech synthesis unit, connected to the response generation unit and the audio output element of the interaction module, and configured to convert the response text generated by the response generation unit into a sound signal, and the audio output element is configured to play speech according to the sound signal generated by the speech synthesis unit.

16. The caregiving robot according to claim 12, wherein the conversation module comprises a response optimization unit, according to personal information of the care recipient, the response optimization unit trains the language model to configure role, wordings, tone and pitch used by the caregiving robot during the conversation.

17. The caregiving robot according to claim 1, further comprising a communication module electrically connected to the control module, wherein the communication module is configured to communicate with a management device of the caregiver and transmit the status report to the management device.

18. The caregiving robot according to claim 1, wherein the control module comprises a storage unit configured to store data required for the caregiving robot to perform the caregiving task, the status report generated by the control module, and/or software executed by the control module.
